(19) United States
(12) Patent Application Publication
(10) Pub. No.: US 2008/0147088 A1
(43) Pub. Date: Jun. 19, 2008

(54) SURGICAL APPARATUS AND METHOD FOR USING THE SAME

(76) Inventor: Chieh-Hsiao Chen, Taichung (TW)

Correspondence Address: PAI PATENT & TRADEMARK LAW FIRM, 1001 FOURTH AVENUE, SUITE 3200, SEATTLE, WA, 98154, US

(21) Appl. No.: 11/939,556

(22) Filed: Nov. 14, 2007

(30) Foreign Application Priority Data
Dec. 13, 2006 (TW) 95146751

(51) Int. Cl. A61B 19/00

(52) U.S. Cl. 606/130

(57) ABSTRACT

A surgical apparatus and a method for using the same are disclosed. The surgical apparatus comprises a inspecting device, a treating device, a parameter generator, a control device, and a monitoring device. The inspecting device inspects a body cavity tissue to determine a treatment area in advance. The treating device is used for treating the body cavity tissue. The parameter generator generates at least one control parameter based on the treatment area. The control device controls the treating device to control the treating device according to the control parameter. The monitoring device monitors if an abnormal situation occurs during surgery.

### RELATED APPLICATIONS

This application claims priority to Taiwan Application Serial Number 95146751, filed Dec. 13, 2006, which is herein incorporated by reference.

### FIELD OF THE INVENTION

This invention relates to a surgical apparatus and a method for using the same, and more particularly, to a surgical apparatus which can treat the body cavity tissue automatically during the surgical procedure and a method for using the same.

### BACKGROUND OF THE INVENTION

During the surgical procedure, it is necessary to use different monitoring apparatus, wherein endoscopy is a minimally invasive diagnostic medical procedure used to assess the interior surfaces of an organ by inserting a tube into the body. An endoscope may have a rigid or flexible tube and not only provide an image for visual inspection and photography, but also enable taking biopsies and retrieval of foreign objects. Endoscopy is the vehicle for minimally invasive surgery.

Many endoscopic procedures are considered to be relatively painless or, at worst, associated with mild discomfort. Most patients tolerate the procedure with only topical anaesthesia of the oropharynx using lignocaine spray. Complications are rare but can include perforation of the organ under inspection with the endoscope or biopsy instrument. If that occurs open surgery may be required to repair the injury

When a surgery (such as prostatic hyperplasia surgery) is performed, doctors have to proceed with the surgery according to the image grabbed by the endoscopy, personal experience or instantaneous determination. However, during the surgical procedure, some unexpected situation may happen due to an improper determination or a man-made mistake. Therefore, accuracy and medical quality of surgery cannot be enhanced.

### SUMMARY OF THE INVENTION

Therefore, an aspect of the present invention is to provide a surgical apparatus and a method for using the same to perform a surgical procedure automatically and enhance accuracy and medical quality thereof.

Another aspect of the present invention is to provide a surgical apparatus and a method for using the same to inspect the body cavity tissue and determine the control parameter thereof before the surgery, thereby preventing the problems of an improper determination or a man-made mistake.

Another aspect of the present invention is to provide a surgical apparatus and a method for using the same to monitor the surgery immediately, thereby preventing an unexpected situation to happen during the surgical procedure.

According to an embodiment of the present invention, the surgical apparatus comprises an inspecting device, a treating device, a parameter generator, a control device, and a monitoring device. The inspecting device inspects a body cavity tissue and determines a treatment area of the surgery before performing the surgical procedure. The treating device comprises a treating member and an actuator. The actuator actuates the treating member to perform a treating action. The parameter generator includes an input element to input the treatment area and generates at least one control parameter based on the treatment area before performing the surgical procedure. The control device is electrically connected to the treating device and the parameter generator to control the treating device according to the control parameter. The monitoring device monitors if an abnormal situation occurs during the surgical procedure, wherein when the abnormal situation occurs, the control device pauses the treating device.

According to another embodiment of the present invention, the method for using a surgical apparatus comprises: before performing the surgical procedure, inspecting a body cavity tissue to determine a treatment area for the surgery by using a inspecting device; before performing the surgical procedure, inputting the treatment area into an input element of a parameter generator to generate at least one control parameter; during the surgical procedure, controlling a treating device to treat the body cavity tissue according to the control parameter by using a control device; and during the surgical procedure, monitoring if an abnormal situation occurs by using a monitoring device, wherein when the abnormal situation occurs, the control device pauses the treating device.

Therefore, with the use of the surgical apparatus and the method for using the same of the present invention, the surgical apparatus can treat the body cavity tissue automatically during the surgical procedure, and the doctor can monitor the surgery immediately to prevent an unexpected situation. Therefore, accuracy and medical quality of surgery are enhanced.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 is a schematic diagram showing a surgical apparatus according to an embodiment of the present invention;

FIG. 2 is an inspection result of a surgical apparatus according to an embodiment of the present invention;

FIG. 3 and FIG. 4 are a plan view showing a surgical apparatus according to an embodiment of the present invention; and

FIG. 5 is a schematic flow diagram showing the method for using a surgical apparatus according to an embodiment of the present invention.

### DETAILED DESCRIPTION OF THE PREFERRED EMBODIMENT

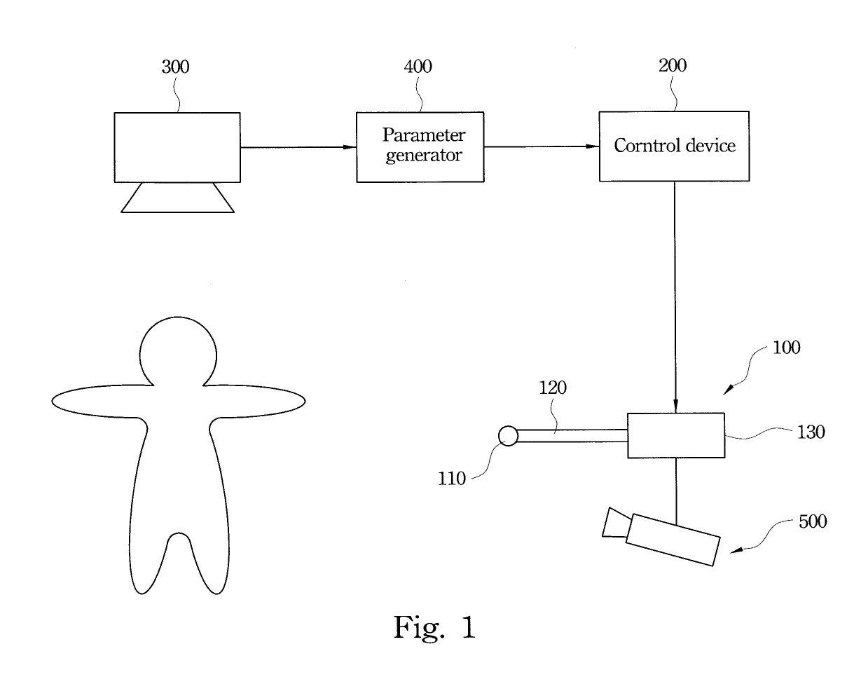

In order to make the illustration of the present invention more explicit and complete, the following description is stated with reference to FIG. 1 through FIG. 5.

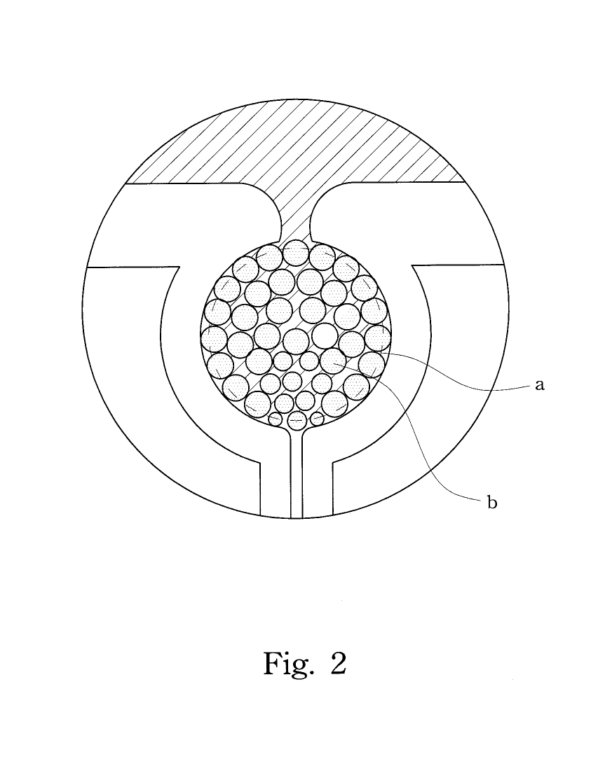

Refer to FIG. 1 and FIG. 2. FIG. 1 is a schematic diagram showing a surgical apparatus according to an embodiment of the present invention. FIG. 2 is an inspection result of a surgical apparatus according to an embodiment of the present invention. The surgical apparatus 100 of the present embodiment can treat (such as resect or photoselective vaporize) a body cavity tissue automatically during a surgical procedure (such as prostatic hyperplasia surgery or esophageal lumpectomy). During the surgical procedure, the doctor merely needs to monitor if an abnormal situation (such as an actual surgical procedure different from the predetermined procedure or a machine problem) occurs, but does not need to perform the procedure personally. The surgical apparatus 100 of the present embodiment comprises a treating device 100, a control device 200, an inspecting device 300, a parameter generator 400 and a monitoring device 500. The control device 200 can control the treating device 100 to treat the body cavity tissue a, such as benign tumor or malignant tumor. The inspecting device 300 inspects the body cavity tissue a before performing the surgical procedure to determine a treatment area b thereof. The treatment area b is inputted into the parameter generator 400 to generate at least one control parameter. The monitoring device 500 monitors if the abnormal situation occurs during the surgical procedure.

Refer to FIG. 1 again. The treating device 100 of the present embodiment includes a treating member 110, a connecting member 120 and an actuator 130. The treating member 110 may be a scalpel, an electrocautery or a laser treating device to perform a treating action, such as resection or photoselective vaporization. In the present embodiment, the treating member 110 may be the laser treating device, preferably a green-light photoselective vaporization apparatus to vaporize the body cavity tissue a, thereby reducing blood lost in surgery and making it easy to convalesce.

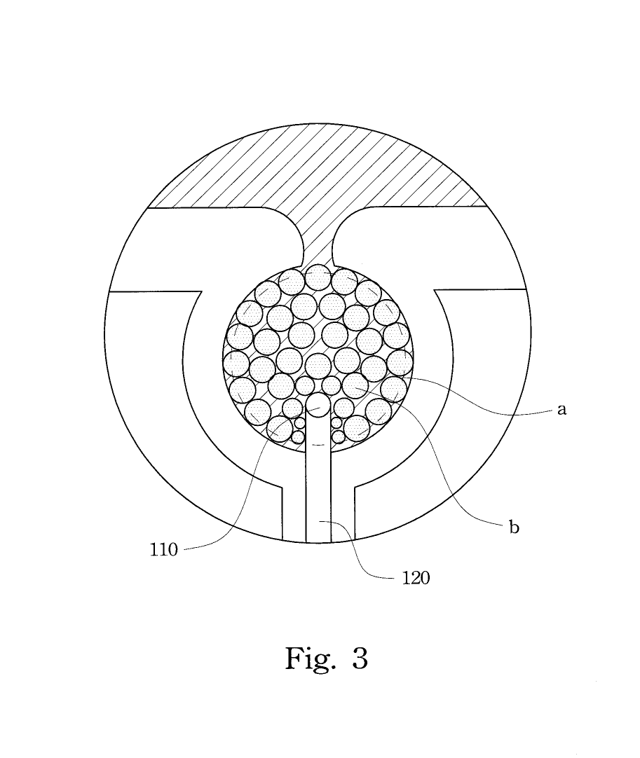

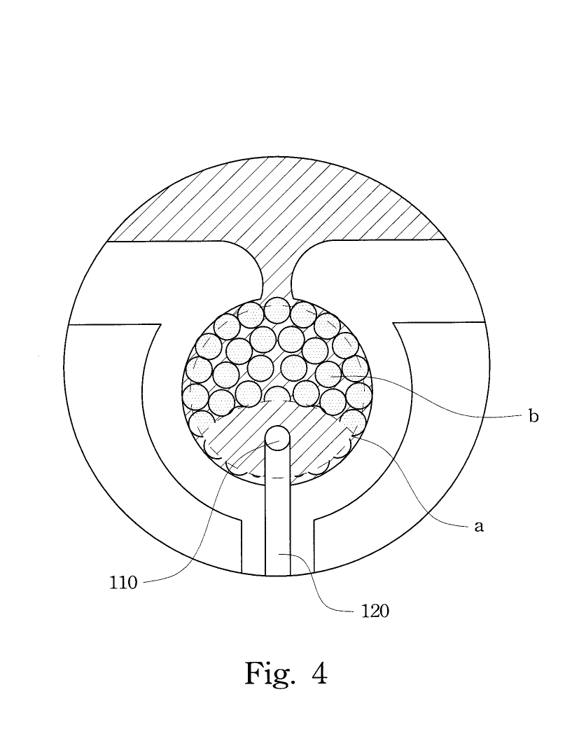

Refer to FIG. 3 and FIG. 4. FIG. 3 and FIG. 4 are a plan view showing a surgical apparatus according to an embodiment of the present invention. The connecting member 120 may be a connecting shaft connected between the treating member 110 and the actuator 130 to allow the actuator 130 to actuate the treating member 110. The actuator 130 may be a motor to actuate the treating member 110 to generate action, such as rotation or motion. Therefore, the treating device 100 can use the treating member 110 to treat the body cavity tissue a.

Refer to FIG. 1 again. The control device 200 of the present embodiment may be a computer device or a programmable control chip electrically connected to the treating device 100 and the parameter generator 400 to control the treating device 100 according to the control parameter. The inspecting device 300 may be a nuclear magnetic resonance imaging apparatus, an X-ray apparatus, a tomography scanner, a supersonic diagnostic apparatus or an endoscope to inspect the body cavity tissue a. The doctor can determine the treatment area b of the body cavity tissue a according the inspection result from the inspecting device 300. The parameter generator 400 may be a programmable control chip electrically connected to the control device 200. The parameter generator 400 includes an input element (not shown) to allow the treatment area b to be inputted, thereby generating the control parameter to the control device 200. The monitoring device 500 may be nuclear magnetic resonance imaging apparatus, a supersonic diagnostic apparatus and an endoscope to monitor the surgery immediately. If an abnormal situation occurs during the surgical procedure, the doctor can use the control device 200 to pause the treating device 100, thereby enhancing medical quality.

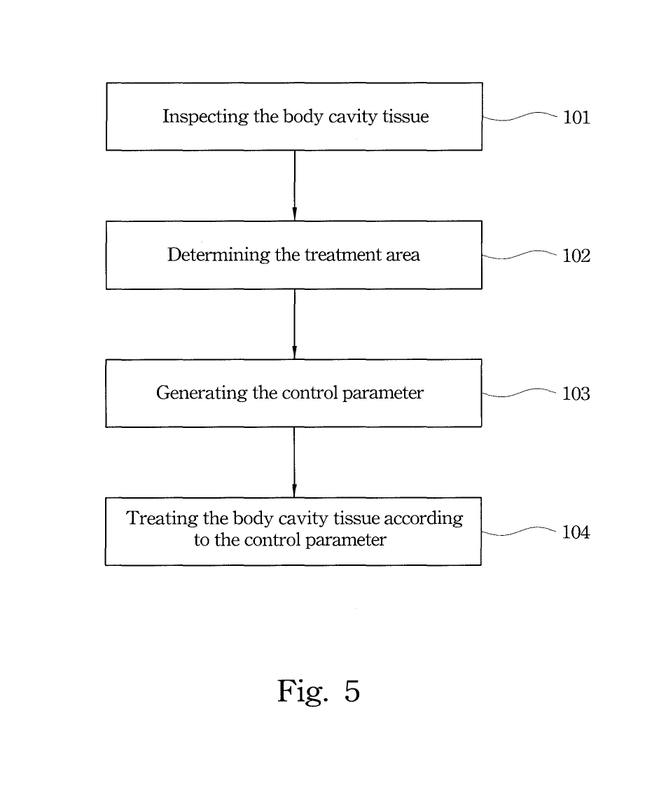

Refer to FIG. 2 and FIG. 5. FIG. 5 is a schematic flow diagram showing the method for using a surgical apparatus according to an embodiment of the present invention. When using the surgical apparatus of the present embodiment, first, the doctor utilizes the inspecting device 300 to inspect the body cavity tissue a to obtain an inspection result before the performing the surgical procedure (step 101). Next, the doctor determines the treatment area b according to the inspection result of the body cavity tissue a (step 102). Next, the treatment area b and the situation of the body cavity tissue a are inputted into the input element of the parameter generator 400 to generate the control parameter transmitted to the control device 200 (step 103). Next, the surgery proceeds, the treating device 100 is controlled by the control device 200 according to the control parameter to treat the body cavity tissue a (step 104), wherein the doctor can use the monitoring device 500 to monitor the surgery immediately. When an abnormal situation occurs, the doctor can use the control device 200 to pause the treating device 100, thereby pausing the surgical procedure to amend the surgery. Therefore, an unexpected situation due to an improper determination or a man-made mistake can be prevented, and accuracy and medical quality of surgery are enhanced.

In the present embodiment, for example, the surgical apparatus is employed in a prostatic hyperplasia surgery. First, the doctor can use the inspecting device 300 to inspect the prostatic tissue and determine the treatment area b of the prostatic tissue, such as determining the area of the prostatic tissue for resecting. Next, the treatment area b and the situation of the prostatic tissue are inputted into the input element of the parameter generator 400 to generate the control parameter (such as treating route, treating speed, direction, or treating time) transmitted to the control device 200. when performing the surgical procedure, the treating device 100 (such as including a green-light photoselective vaporization apparatus) is controlled by the control device 200 according to the control parameter to treat the prostatic tissue (i.e. resecting the prostatic hyperplasia tissue). During the surgical procedure, the doctor merely needs to use the monitoring device 500 to monitor the surgery immediately. When an abnormal situation occurs, the doctor can use the control device 200 to pause the surgical procedure.

Therefore, the surgical apparatus of the present embodiment can inspect the body cavity tissue before the surgical procedure to determine the treatment area and how to operate the surgery (the control parameter), and the doctor can monitor the surgery immediately to prevent an unexpected situation. Therefore, accuracy and medical quality of surgery are enhanced.

As is understood by a person skilled in the art, the foregoing embodiments of the present invention are strengths of the present invention rather than limiting of the present invention. It is intended to cover various modifications and similar arrangements included within the spirit and scope of the appended claims, the scope of which should be accorded the broadest interpretation so as to encompass all such modifications and similar structures.

What is claimed is:

1. A surgical apparatus for performing a surgical procedure, comprising:
an inspecting device to inspect a body cavity tissue and determine a treatment area of the surgery before performing the surgical procedure;
a treating device, comprising:
a treating member to treat the body cavity tissue; and
an actuator to actuate the treating member to perform a treating action;
a parameter generator including an input element to input the treatment area and generating at least one control parameter based on the treatment area before performing the surgical procedure;
a control device electrically connected to the treating device and the parameter generator to control the treating device according to the control parameter; and
a monitoring device monitoring if an abnormal situation occurs during the surgical procedure, wherein when the abnormal situation occurs, the control device pauses the treating device.

2. The surgical apparatus as claimed in claim 1, wherein the treating device further comprises:
a connecting member connected between the treating member and the actuator.

3. The surgical apparatus as claimed in claim 1, wherein the monitoring device is selected from a group consisting of a nuclear magnetic resonance imaging apparatus, a supersonic diagnostic apparatus and an endoscope.

4. The surgical apparatus as claimed in claim 1, wherein the inspecting device is selected from a group consisting of a nuclear magnetic resonance imaging apparatus, an X-ray apparatus, a tomography scanner, a supersonic diagnostic apparatus and an endoscope.

5. The surgical apparatus as claimed in claim 1, wherein the treating member is selected from a group consisting of a scalpel, an electrocautery and a laser treating device.

6. The surgical apparatus as claimed in claim 5, wherein the treating member is a green-light photoselective vaporization apparatus.

7. The surgical apparatus as claimed in claim 1, wherein the control device is a computer device.

8. The surgical apparatus as claimed in claim 1, wherein the control device is a programmable control chip.

9. The surgical apparatus as claimed in claim 1, wherein the actuator is a motor.

10. A method for using a surgical apparatus to perform a surgical procedure, comprising:
before performing the surgical procedure, inspecting a body cavity tissue to determine a treatment area of the surgery by using a inspecting device;
before performing the surgical procedure, inputting the treatment area into an input element of a parameter generator to generate at least one control parameter;
during the surgical procedure, controlling a treating device to treat the body cavity tissue according to the control parameter by using a control device; and
during the surgical procedure, monitoring if an abnormal situation occurs by using a monitoring device, wherein when an abnormal situation occurs, the control device pauses the treating device.

11. The method for using a surgical apparatus as claimed in claim 10, wherein the monitoring device is selected from a group consisting of a nuclear magnetic resonance imaging apparatus, a supersonic diagnostic apparatus and an endoscope.

12. The method for using a surgical apparatus as claimed in claim 10, wherein the treating device, comprising:
a treating member to treat the body cavity tissue; and
an actuator to actuate the treating member to perform a treating action.

13. The method for using a surgical apparatus as claimed in claim 12, wherein the treating device further comprises:
a connecting member connected between the treating member and the actuator.

14. The method for using a surgical apparatus as claimed in claim 12, wherein the treating member is selected from a group consisting of a scalpel, an electrocautery, a laser treating device.

15. The method for using a surgical apparatus as claimed in claim 12, wherein the actuator is a motor.

16. The method for using a surgical apparatus as claimed in claim 12, wherein the treating action is resection.

17. The method for using a surgical apparatus as claimed in claim 12, wherein the treating action is photoselective vaporization.

18. The method for using a surgical apparatus as claimed in claim 10, wherein the inspecting device is selected from a group consisting of a nuclear magnetic resonance imaging apparatus, an X-ray apparatus, a tomography scanner, a supersonic diagnostic apparatus and an endoscope.

19. The method for using a surgical apparatus as claimed in claim 10, wherein the control device is a computer device.

20. The method for using a surgical apparatus as claimed in claim 10, wherein the control device is a programmable control chip.
